(19) United States
(12) Patent Application Publication
(10) Pub. No.: US 2020/0126662 A1
(43) Pub. Date: Apr. 23, 2020

(54) METHOD, DEVICE, AND APPARATUS FOR DETECTING DISEASE PROBABILITY, AND COMPUTER-READABLE STORAGE MEDIUM

(71) Applicant: Ping An Technology (Shenzhen) Co., Ltd., Guangdong (CN)

(72) Inventors: Feifei Li, Shenzhen, Guangdong (CN); Liang Xu, Shenzhen, Guangdong (CN); Jing Xiao, Shenzhen, Guangdong (CN)

(73) Assignees: Ping An Technology (Shenzhen) Co., Ltd., Shenzhen, Guangdong (CN); Ping An Technology (Shenzhen) Co., Ltd., Shenzhen, Guangdong (CN)

(21) Appl. No.: 16/305,884

(22) PCT Filed: Jan. 31, 2018
(86) PCT No.: PCT/CN2018/074808

§ 371 Date: Nov. 29, 2018

(30) Foreign Application Priority Data
Feb. 20, 2017 (CN) 201710095020.5

(51) Int. Cl. G16H 50/20; G16H 70/60

(52) U.S. Cl. CPC G16H 50/20; G16H 70/60

(57) ABSTRACT

The present disclosure discloses a method for detecting disease probability which includes: collecting each datum associated with a user, and performing feature processing to each collected datum; constructing a multi-dimensional data set according to each datum after being feature processed; performing random sampling on the multi-dimensional data set to divide into a test set and a training set; building a model based on the training set to obtain a regression decision tree; testing the regression decision tree according to the test set, to calculate the disease probability of the user. The present disclosure further discloses a device, an apparatus for detecting disease probability, and a computer-readable storage medium.

This application claims priority to Chinese Patent Application No. 201710095020.5, filed with the Chinese Patent Office on Feb. 20, 2017 and entitled “Method and Device for Detecting Disease Probability”, which is incorporated herein by reference in its entirety.

### FIELD

The present disclosure relates to the field of disease information processing, and more particularly to a method, a device, and an apparatus for detecting disease probability, and a computer-readable storage medium.

### BACKGROUND

Conventional disease probability detecting, such as cancer incidence detecting, is commonly based on biology, genomics, and physical examination results, which is complexly to be carried out. This detecting method requires an accurate data source, and after the data source being obtained, it still needs a long time to analyze and process the data source to obtain a detection result. In addition, the approach to obtain the data source is also complicated, as a result the cost for disease detecting is high. Therefore, the existing detecting for disease probability cannot detect disease probability quickly, and the cost for the disease probability detecting is also high.

### SUMMARY

The present disclosure is to provide a method, a device, and an apparatus for detecting disease probability, and computer-readable storage medium, which aims to solve the technical problems of that the disease probability detection takes a long time and a high cost in the prior art.

In order to achieve the above aim, the present disclosure provides a method for detecting disease probability which includes:

collecting each datum associated with a user, and performing feature processing to each collected datum;

constructing a multi-dimensional data set according to each datum after being feature processed;

performing random sampling on the multi-dimensional data set to divide into a test set and a training set;

building a model based on the training set to obtain a regression decision tree;

testing the regression decision tree according to the test set, to calculate the disease probability of the user.

Furthermore, in order to achieve the above aim, the present disclosure provides a device for detecting disease probability which includes:

a processing module, configured for collecting each datum associated with a user, and performing feature processing to each collected datum;

a constructing module, configured for constructing a multi-dimensional data set according to each datum after being feature processed;

a dividing module, configured for performing random sampling on the multi-dimensional data set to divide into a test set and a training set;

a building module, configured for building a model based on the training set to obtain a regression decision tree;

a calculating module, configured for testing the regression decision tree according to the test set, to calculate the disease probability of the user.

Furthermore, in order to achieve the above aim, the present disclosure provides an apparatus for detecting disease probability which includes: a processor, a memory which stores a disease probability detecting program; wherein the processor is configured for executing the disease probability detecting program to perform the aforesaid operations of the disease probability detecting method.

Furthermore, in order to achieve the above aim, the present disclosure provides a computer-readable storage medium which stores a disease probability detecting program, above operations of the method for detecting disease probability are performed when the disease probability detecting program executed by the processor.

The method and device provided in this present disclosure, first collect each datum associated with the user, and perform feature processing to each collected datum; then construct the multi-dimensional data set according to each datum after being feature processed; and then perform random sampling to the multi-dimensional data set to divide into the test set and the training set; afterwards build the model based on the training set to obtain the regression decision tree; finally test the regression decision tree according to the test set, to calculate the disease probability of the user. The present disclosure builds the model through the collected data, and finally calculates the disease probability of the user according to the built model without detecting the disease probability by means of physical examination, so that the detecting efficiency of the disease probability is relatively high, and the cost of disease probability detection is relatively low.

### BRIEF DESCRIPTION OF THE DRAWINGS

FIG. 1 is a flowchart illustrating a first embodiment of a method for detecting disease probability according to the present disclosure.

FIG. 2 is a detailed flowchart illustrating S10 in FIG. 1.

FIG. 3 is a detailed flowchart illustrating S20 in FIG. 1.

FIG. 4 is a detailed flowchart illustrating S50 in FIG. 1.

FIG. 5 is a block diagram illustrating a first embodiment of a device for detecting disease probability according to the present disclosure.

FIG. 6 is a detailed block diagram illustrating a processing module 10 in FIG. 5.

FIG. 7 is a detailed block diagram illustrating a constructing module 20 in FIG. 5.

FIG. 8 is a detailed block diagram illustrating a calculating module 50 in FIG. 5.

FIG. 9 is a schematic structural diagram of a hardware operating environment device according to an embodiment of the present disclosure.

Various implementations, functional features, and advantages of the present disclosure will now be described in further detail with reference to the accompanying drawings and some illustrative embodiments.

### DETAILED DESCRIPTION OF THE EMBODIMENTS

It is to be understood that, the specific embodiments described herein portrays merely some illustrative embodiments of the present disclosure, and are not intended to limit the patentable scope of the present disclosure.

The solution of the embodiments of the present disclosure mainly is: first collect each datum associated with the user, and perform feature processing to each collected datum; then construct the multi-dimensional data set according to each datum after being feature processed; and then perform random sampling on the multi-dimensional data set to divide into the test set and the training set; afterwards build the model based on the training set to obtain the regression decision tree; finally test the regression decision tree according to the test set, to calculate the disease probability of the user. The technical problems of that a physical examination and laboratory test are required, the disease probability detection takes a long time and a high cost in the prior art are solved.

It should be understood that conventionally in disease detecting, the approach to obtain data source is relatively complicated, and it is impossible to detect the disease probability quickly for each ordinary user, and the conventional method is also difficult to implement in the insurance industry.

Based on the problems existing in the prior art, the present disclosure provides a method for detecting disease probability.

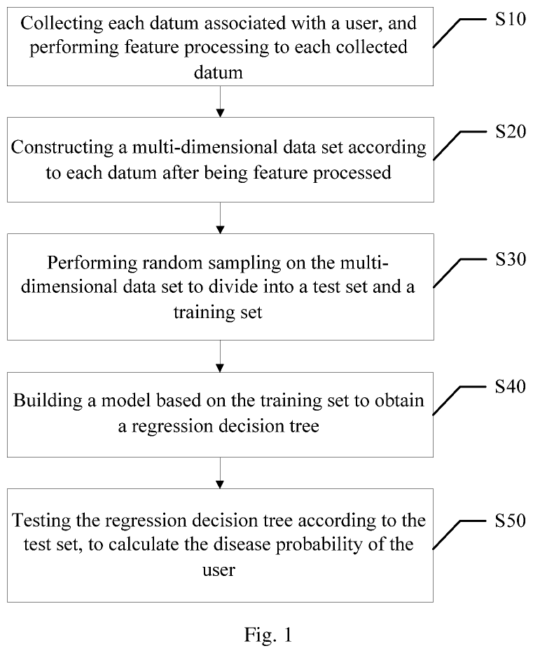

Referring to FIG. 1, FIG. 1 is a flowchart illustrating a first embodiment of the method for detecting disease probability according to the present disclosure.

In this embodiment, the method for detecting disease probability includes:

Collecting each datum associated with a user, and performing feature processing to each collected datum; constructing a multi-dimensional data set according to each datum after being feature processed; performing random sampling on the multi-dimensional data set to divide into a test set and a training set; building a model based on the training set to obtain a regression decision tree; testing the regression decision tree according to the test set, to calculate the disease probability of the user.

The following are the specific steps to gradually implement the disease probability detection in this embodiment:

S10, collecting each datum associated with a user, and performing feature processing to each collected datum;

In this embodiment, the method for detecting disease probability is preferably applied to an insurance system. It can be understood that the user may report data of health information related to medical examination or some personal behavior information to the insurance system before the insurance is insured, a comprehensive analysis is performed by the insurance system to detect the user's disease probability, afterwards to determine whether to insure. Therefore, collecting each datum associated with the user in the database is actually collecting each datum associated with the user in the database corresponding to the insurance system. In this embodiment, the data includes behavior information and health information, and the behavior information and the health information represent information in different dimensions.

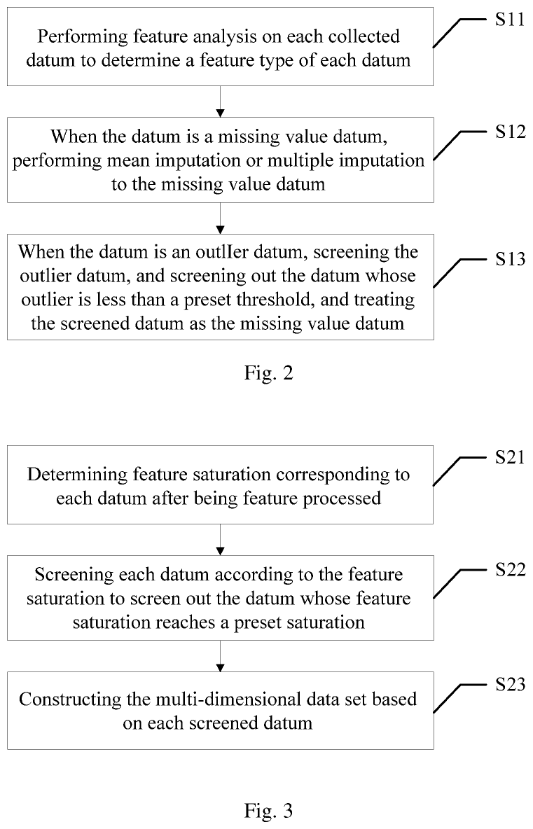

After collecting each datum associated with the user, perform feature processing to each collected datum. Specifically, referring to FIG. 2, S10 includes:

S11, performing feature analysis on each collected datum to determine a feature type of each datum;

S12, when the datum is a missing value datum, performing mean imputation or multiple imputation to the missing value datum;

S13, when the datum is an outlier datum, screening the outlier datum, and screening out the datum whose outlier is less than a preset threshold, and treating the screened datum as the missing value datum.

That is, after each datum associated with the user is collected, the collected datum is subjected to feature analysis to determine the feature type of each datum. In this embodiment, the feature types of the datum include feature types such as an outlier and a missing value. After determining the feature type of each datum, if the datum is a missing value datum, perform mean imputation or multiple imputation to the missing value datum, and specifically, adopt which interpolation processing method according to actual conditions.

In this embodiment, the mean interpolation includes two modes: 1) one is using an average value to perform interpolation; 2) the other one is using the mode to perform interpolation. Specifically, first the attribute of the datum is divided into interval data type and non-interval data type. If the missing value belongs to the interval data type, the missing value is interpolated with the average value of existing values with the attribute; if the missing value is non-interval data type, according to the principle of the mode in statistics, use the mode (that is, the value with the highest frequency of occurrence) with the attribute to fill in the missing value.

The multiple imputation (MI) regards that the value to be interpolated is random and its value is derived from observed values. In practice, usually the value to be interpolated is estimated, and then is added different noises to form multiple sets of optional interpolation values. The multiple imputation is divided into three steps: 1. Generate a set of possible interpolation values for each null value, which reflect the uncertainty of a non-response model; each value can be used to interpolate the missing value in the data set, resulting in several complete data sets. 2. Each data set of interpolation is statistically analyzed using a statistical method for the complete data set. 3. The result from each respective data set of interpolation is selected according to a scoring function to generate a final imputation value.

For example, currently there is a data group, including three variables Y1, Y2, Y3, and their joint distribution is normal distribution. The data are processed into three groups. Group A maintains the original data, and Group B only lacks Y3, Group C lacks Y1 and Y2. When performing multiple imputation, no processing would be performed for group A, a set of estimated values of Y3 are generated for group B (performing regression for Y3 with respect to Y1, Y2), and a set of estimated values in pair of Y1 and Y2 are generated for group C (performing regression for Y1 and Y2 with respect to Y3). When using multiple imputation, Group A will not be processed, for Group B and C, entire samples will be randomly selected to form m groups (m is the optional m groups of interpolation values), and as long as the number of cases in each group can effectively estimate the parameters. Estimate the distribution of attributes with missing values, and then based on the m groups of observation values, respectively generate m groups of estimated values about the parameters for the m groups of samples, provide the corresponding predicted value, the estimation method used here is Maximum Likelihood Estimate method, the specific implementation algorithm in the computer is the Expectation-maximization algorithm (EM). A set of Y3 values are estimated for Group B, and a set of (Y1, Y2) are estimated for Group C premised on the joint distribution of Y1, Y2, and Y3 being normal distribution.

By the above means, mean value interpolation or multiple interpolation can be performed for the missing value data.

Certainly, if the datum is found to be an outlier datum, screen the outlier datum, to screen out the datum whose outlier is less than a preset threshold. The preset threshold is defined according to specific situations. After screening out the datum whose outlier is less than the preset threshold, the screened datum can be regarded as the missing value datum, and the processing method for the missing value data has been described above, details are not described herein again.

It should be understood that, in this embodiment, the interpolation processing of the datum is equivalent to filling up the datum with the missing value. The reason to fill up the content is that the data collected from the database may have some information which is not completely filled, if calculating the disease probability subsequently, it may not be accurate. Therefore, in this embodiment, to fill up the datum with missing value can improve the saturation of the data, and ensure the accuracy of the subsequent disease probability calculation. The screening of outliers is to eliminate data with relatively serious abnormalities to prevent the impact on disease probability detection results.

S20, constructing a multi-dimensional data set according to each datum after being feature processed;

After performing feature processing on each collected datum, a multi-dimensional data set is constructed according to each datum after being feature processed. It can be understood that the above has disclosed that the data with missing values are filled, but the filled data may not meet the requirements of saturation. If the data are used for subsequent calculation, the accuracy of the disease probability may still be lowered. Therefore, in the present embodiment, in order to improve the accuracy of the disease probability calculation, referring to FIG. 3, S20 includes:

S21, determining feature saturation corresponding to each datum after being feature processed;

S22: screening each datum according to the feature saturation to screen out each datum whose feature saturation reaches a preset saturation degree;

S23, constructing a multi-dimensional data set according to each selected datum.

That is, after performing feature processing to each collected datum, first determine the feature saturation corresponding to each datum after being feature processed; then screening each datum according to the feature saturation to screen out each datum whose feature saturation reaches a preset saturation degree; at last construct the multi-dimensional data set according to each selected datum. It is equivalent to cleaning the collected data to screen out the data that meets the requirements, so as to ensure that the subsequent disease probability calculation is relatively accurate.

S30, performing random sampling on the multi-dimensional data set to divide into a test set and a training set;

That is, after constructing the multi-dimensional data set, perform random sampling on the multi-dimensional data set to divide into a test set and a training set. In this embodiment, the number of the test set and the training set is not limited, and is set according to specific situations, but the number of the training set is required to be higher than the number of the test set, for example, the training set is divided into 70%, and the test set is divided into 30%.

S40, building a model based on the training set to obtain a regression decision tree;

Based on the training set, the model is built, the regression decision tree is obtained. In this embodiment, the way to build the model according to the training set is consistent with the way to build the model according to the existing data, and details are not described here.

S50, testing the regression decision tree according to the test set, to calculate the disease probability of the user.

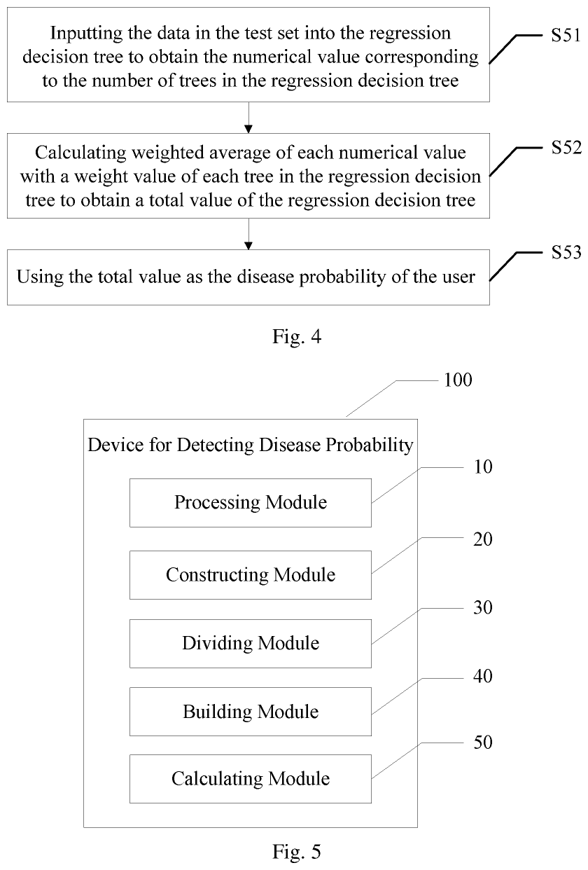

After obtaining the regression decision tree, test the regression decision tree according to the test set, to calculate the disease probability of the user. Referring to FIG. 4, S50 includes:

S51, inputting the data in the test set into the regression decision tree to obtain the numerical value corresponding to the number of trees in the regression decision tree;

S52, calculating weighted average of each numerical value with a weight value of each tree in the regression decision tree to obtain a total value of the regression decision tree;

S53, using the total value as the disease probability of the user.

That is, the regression decision tree is tested according to the test set to calculate the disease probability of the user, substantially inputting the data of the test set into the regression decision tree, and then obtain the corresponding number of values according to the number of trees in the regression decision tree. For example, the number of trees in the current regression decision tree is 3000-5000, and the number of obtained values is also the number of trees 3000-5000. Due to the weight value of the respective tree in the regression decision tree is preset, then after obtaining the numerical value corresponding to the number of trees in the regression decision tree, calculate weighted average of each numerical value with the weight value of each tree in the regression decision tree to obtain the total value of the regression decision tree. For example, the regression decision tree has four trees with weights of 0.3, 0.15, 0.2, and 0.35, and the obtained respective values according to the number of trees in the regression decision tree are A, B, C, and D, then the total value Q=0.3*A+0.15*B+0.2*C+0.35*D. This total value is the disease probability of the user.

In this embodiment, it is equivalent to outputting the predicted result of the model to obtain the disease probability of the user though the regression decision tree model for the user whose disease condition is unknown.

The method and device provided in this present disclosure, first collect each datum associated with the user, and perform feature processing to each collected datum; then construct the multi-dimensional data set according to each datum after being feature processed; and then perform random sampling on the multi-dimensional data set to divide into the test set and the training set; afterwards build the model based on the training set to obtain the regression decision tree; finally test the regression decision tree according to the test set, to calculate the disease probability of the user. The present disclosure builds the model through the collected data, and finally calculates the disease probability of the user according to the built model without detecting the disease probability by means of physical examination, so that the detecting efficiency of the disease probability is relatively high, and the cost of disease probability detecting is relatively low.

It should be noted that those skilled in the art may understand that all or part of the operations of the above embodiments may be performed by hardware, or may be performed through a program to instruct related hardware to execute, and the program may be stored in a computer-readable storage medium, the above-mentioned storage medium may be a read only memory, a magnetic disk or an optical disk or the like.

The present disclosure further provides a device for detecting disease probability.

Referring to FIG. 5, FIG. 5 is a block diagram illustrating a first embodiment of the device for detecting disease probability 100 according to the present disclosure.

It should be emphasized that, for those skilled in the art, the block diagram shown in FIG. 5 is merely an exemplary diagram of a preferred embodiment, and the functional module of the device for detecting disease probability 100 shown in FIG. 5 can be easily supplemented by a new functional module by those skilled in the art. The name of each functional module is a custom name, is merely for assisting in understanding each program function block in the device for detecting disease probability 100, not used to limit the technical solution of the present disclosure. The core of the present disclosure is the function to be achieved by the functional module with each custom name.

In this embodiment, the device for detecting disease probability 100 includes:

a processing module 10, configured for collecting each datum associated with a user, and performing feature processing to each collected datum;

a constructing module 20, configured for constructing a multi-dimensional data set according to each datum after being feature processed;

a dividing module 30, configured for performing random sampling on the multi-dimensional data set to divide into a test set and a training set;

a building module 40, configured for building a model based on the training set to obtain a regression decision tree;

a calculating module 50, configured for testing the regression decision tree according to the test set, to calculate the disease probability of the user.

In this embodiment, the device for detecting disease probability is preferably applied to an insurance system. It can be understood that the user may report data of health information related to medical examination or some personal behavior information to the insurance system before the insurance is insured, a comprehensive analysis is performed by the insurance system to detect the user's disease probability, afterwards to determine whether to insure. Therefore, the processing module 10 collecting each datum associated with the user in the database is actually collecting each datum associated with the user in the database corresponding to the insurance system. In this embodiment, the data includes behavior information and health information, and the behavior information and the health information represent information in different dimensions.

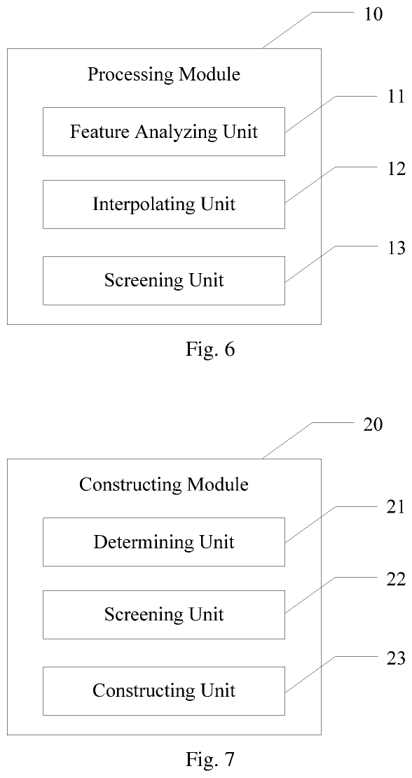

After collecting each datum associated with the user, the processing module 10 performs feature processing to each collected datum. Specifically, referring to FIG. 6, the processing module 10 includes:

a feature analyzing unit 11, configured for perform feature analysis on each collected datum to determine a feature type of each datum;

an interpolating unit 12, configured for when the datum is a missing value datum, performing mean imputation or multiple imputation to the missing value datum;

a screening unit 13, configured for when the datum is an outlier datum, screening the outlier datum, and screening out the datum whose outlier is less than a preset threshold, and treating the screened datum as the missing value datum.

That is, after each datum associated with the user is collected, the collected datum is subjected to feature analysis to determine the feature type of each datum by the feature analyzing unit 11. In this embodiment, the feature types of the datum include feature types such as an outlier and a missing value. After determining the feature type of each datum, if the datum is a missing value datum, perform mean imputation or multiple imputation to the missing value datum by the interpolating unit 12, and specifically, according to actual conditions, adopt which interpolation processing method.

In this embodiment, the mean interpolation includes two modes: 1) one is using an average value to perform interpolation; 2) the other one is using the mode to perform interpolation. Specifically, first the attribute of the datum is divided into interval data type and non-interval data type. If the missing value belongs to the interval data type, the missing value is interpolated with the average value of existing values with the attribute; if the missing value is non-interval data type, according to the principle of the mode in statistics, use the mode (that is, the value with the highest frequency of occurrence) with the attribute to fill in the missing value.

The multiple imputation (MI) regards that the value to be interpolated is random and its value is derived from observed values. In practice, usually the value to be interpolated is estimated, and then is added different noises to form multiple sets of optional interpolation values. The multiple imputation is divided into three steps: 1. Generate a set of possible interpolation values for each null value, which reflect the uncertainty of a non-response model; each value can be used to interpolate the missing value in the data set, resulting in several complete data sets. 2. Each data set of interpolation is statistically analyzed using a statistical method for the complete data set. 3. The result from each respective data set of interpolation is selected according to a scoring function to generate a final imputation value.

For example, currently there is a data group, including three variables Y1, Y2, Y3, and their joint distribution is normal distribution. The data are processed into three groups. Group A maintains the original data, and Group B only lacks Y3, Group C lacks Y1 and Y2. When performing multiple imputation, no processing would be performed for group A, a set of estimated values of Y3 are generated for group B (performing regression for Y3 with respect to Y1, Y2), and a set of estimated values in pair of Y1 and Y2 are generated for group C (performing regression for Y1 and Y2 with respect to Y3). When using multiple imputation, Group A will not be processed, for Group B and C, entire samples will be randomly selected to form m groups (m is the optional m groups of interpolation values), and as long as the number of cases in each group can effectively estimate the parameters. Estimate the distribution of attributes with missing values, and then based on the m groups of observation values, respectively generate m groups of estimated values about the parameters for the m groups of samples, provide the corresponding predicted value, the estimation method used here is Maximum Likelihood Estimate method, the specific implementation algorithm in the computer is the Expectation-maximization algorithm (EM). A set of Y3 values are estimated for Group B, and a set of (Y1, Y2) are estimated for Group C premised on the joint distribution of Y1, Y2, and Y3 being normal distribution.

By the above means, mean value interpolation or multiple interpolation can be performed for the missing value data.

Certainly, if the datum is found to be an outlier datum, screen the outlier datum by the screening unit 13, to screen out the datum whose outlier is less than a preset threshold. The preset threshold is defined according to specific situations. After screening out the datum whose outlier is less than the preset threshold, the screened datum can be regarded as the missing value datum, and the processing method for the missing value data has been described above, details are not described herein again.

It should be understood that, in this embodiment, the interpolation processing of the datum is equivalent to filling up the datum with the missing value. The reason to fill up the content is that the data collected from the database may have some information which is not completely filled, if calculating the disease probability subsequently, it may not be accurate. Therefore, in this embodiment, to fill up the datum with missing value can improve the saturation of the data, and ensure the accuracy of the subsequent disease probability calculation. The screening of outliers is to eliminate data with relatively serious abnormalities to prevent the impact on disease probability detection results.

After the processing module 10 performs feature processing on each collected datum, a multi-dimensional data set is constructed according to each datum after being feature processed by the constructing module 20. It can be understood that the above has disclosed that the data with missing values are filled, but the filled data may not meet the requirements of saturation. If the data are used for subsequent calculation, the accuracy of the disease probability may still be lowered. Therefore, in the present embodiment, in order to improve the accuracy of the disease probability calculation, referring to FIG. 7, the constructing module 20 includes:

a determining unit 21, configured for determining feature saturation corresponding to each datum after being feature processed;

a screening unit 22, configured for screening each datum according to the feature saturation to screen out the datum whose feature saturation reaches a preset saturation;

a constructing unit 23, configured for constructing the multi-dimensional data set based on each screened datum.

That is, after performing feature processing to each collected datum by the processing module 10, first the determining unit 21 determines the feature saturation corresponding to each datum after being feature processed; then the screening unit 22 screens each datum according to the feature saturation to filter out each datum whose feature saturation reaches a preset saturation degree; at last the constructing unit 23 constructs the multi-dimensional data set according to each selected datum. It is equivalent to cleaning the collected data to screen out the data that meets the requirements, so as to ensure that the subsequent disease probability calculation is relatively accurate.

In this embodiment, after the constructing module 20 constructs the multi-dimensional data set, the dividing module 30 perform random sampling on the multi-dimensional data set to divide into a test set and a training set. In this embodiment, the number of the test set and the training set is not limited, and is set according to specific situations, but the number of the training set is required to be higher than the number of the test set, for example, the training set is divided into 70%, and the test set is divided into 30%.

The building module 40 builds the model based on the training set, the regression decision tree is obtained. In this embodiment, the way to build the model according to the training set is consistent with the way to build the model according to the existing data, and details are not described here.

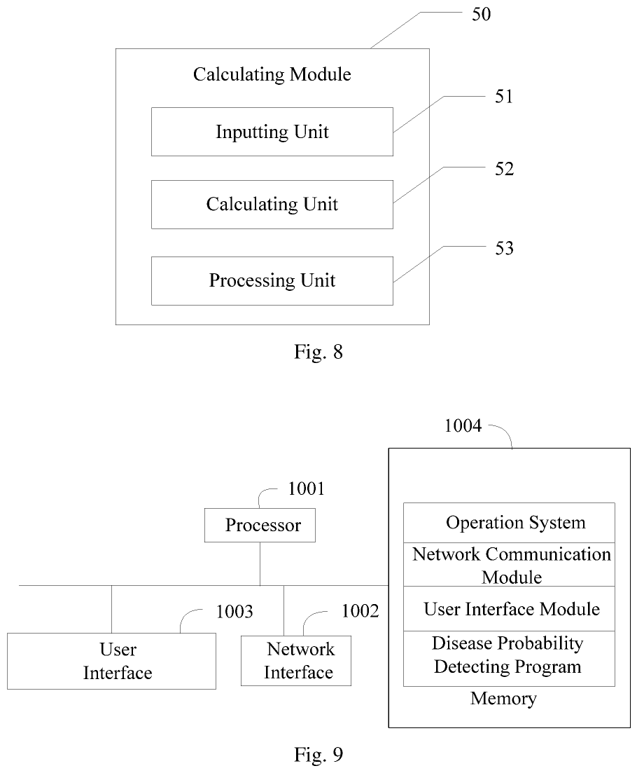

After obtaining the regression decision tree, the calculating module 50 tests the regression decision tree according to the test set, to calculate the disease probability of the user. Referring to FIG. 8, the calculating module 50 includes:

an inputting unit 51, configured for inputting the data in the test set into the regression decision tree to obtain the numerical value corresponding to the number of trees in the regression decision tree;

a calculating unit 52, configured for calculating weighted average of each numerical value with a weight value of each tree in the regression decision tree to obtain a total value of the regression decision tree;

a processing unit 53, configured for using the total value as the disease probability of the user.

That is, the regression decision tree is tested according to the test set to calculate the disease probability of the user by the calculating module 50, substantially is inputting the data of the test set into the regression decision tree by the inputting unit 51, and then obtain the numerical value corresponding to the number of trees in the regression decision tree. For example, the number of trees in the current regression decision tree is 3000-5000, and the number of obtained values is also the number of trees 3000-5000. Due to the weight value of the respective tree in the regression decision tree is preset, then after obtaining the numerical value corresponding to the number of trees in the regression decision tree, the calculating unit 52 calculates weighted average of each value with the weight value of each tree in the regression decision tree to obtain the total value of the regression decision tree. For example, the regression decision tree has four trees with weights of 0.3, 0.15, 0.2, and 0.35, and the obtained respective values according to the number of trees in the regression decision tree are A, B, C, and D, then the total value Q=0.3*A+0.15*B+0.2*C+0.35*D. This total value is the disease probability of the user.

In this embodiment, it is equivalent to outputting the predicted result of the model to obtain the disease probability of the user though the regression decision tree model for the user whose disease condition is unknown.

The method and device provided in this present disclosure, first collect each datum associated with the user, and perform feature processing to each collected datum; then construct the multi-dimensional data set according to each datum after being feature processed; and then perform random sampling on the multi-dimensional data set to divide into the test set and the training set; afterwards build the model based on the training set to obtain the regression decision tree; finally test the regression decision tree according to the test set, to calculate the disease probability of the user. The present disclosure builds the model through the collected data, and finally calculates the disease probability of the user according to the built model without detecting the disease probability by means of physical examination, so that the detecting efficiency of the disease probability is relatively high, and the cost of disease probability detecting is relatively low.

It should be noted that, regard to hardware implementation, the foregoing processing module 10, the constructing module 20, the dividing module 30, the building module 40, the calculating module 50, and the like may be embedded in the disease probability detection device or independent from the disease probability detection device, or stored in the memory of the disease probability detection device in the form of software, so as to be called by the processor to perform the operations corresponding to the above respective modules. The processor can be a central processing unit (CPU), a microprocessor, a microcontroller, or the like.

Referring to FIG. 9, FIG. 9 is a schematic structural diagram of a hardware operating environment device according to an embodiment of the present disclosure.

The device for detecting disease probability in the embodiment of the present disclosure may be a PC, or may be a terminal device such as a smart phone, a tablet computer, or a portable computer.

As shown in FIG. 9, the device for detecting disease probability may include a processor 1001, such as a CPU, a network interface 1002, a user interface 1003, and a memory 1004. Connection communication between these components can be achieved via a communication bus. The network interface 1002 may optionally include a standard wired interface (for connecting to a wired network), a wireless interface (such as a WI-FI interface, a Bluetooth interface, an infrared interface, etc., for connecting to a wireless network). The user interface 1003 may include a display, an input unit such as a keyboard, the user interface 1003 may optionally further include a standard wired interface (such as for connecting with a wired keyboard, a wired mouse) and a wireless interface (such as for connecting with a wireless keyboard, wireless mouse). The memory 1004 may be a high-speed RAM memory or a non-volatile memory such as a disk memory. Optionally the memory 1004 may be a storage device that is separate from the said processor 1001.

Optionally, the device for detecting disease probability may also include a camera, an RF (Radio Frequency) circuit, a sensor, an audio circuit, a WiFi module, and the like.

It could be understood by those skilled in the art that the structure of the device for detecting disease probability shown in FIG. 9 does not constitute a limitation for the device for detecting disease probability, and may include more or less components than those illustrated, or may combine some components, or different part layout.

As shown in FIG. 9, the memory 1004 as a computer storage medium may include an operating system, a network communication module, a user interface module, and a program for detecting disease probability. The operating system is a program for managing and controlling hardware and software resources of the device for detecting disease probability, and for supporting operations of the network communication module, the user interface module, the program for detecting disease probability, and other programs or software; the network communication module is configured for managing and controlling the network interface 1002; the user interface module is configured for managing and controlling the user interface 1003.

In the device for detecting disease probability shown in FIG. 9, the processor 1001 can be used to execute the program for detecting disease probability stored in the memory 1004 to implement the respective operations of the method detecting disease probability as described above.

The present disclosure provides a computer-readable storage medium for detecting disease probability which stores a disease probability detecting program, above operations of the method for detecting disease probability are performed when the disease probability detecting program executed by the processor.

It should be noted that, throughout this disclosure, the terms “include”, “comprise” or any other variations thereof are intended to encompass non-exclusive inclusions, so that a process, method, article, or system that includes a series of elements would include not only those elements, but it may further include other elements that are not explicitly listed or elements that are inherent to such processes, methods, articles, or systems. In the absence of extra limitations, an element defined by the phrase “includes a . . . ” does not exclude the presence of additional identical elements in this process, method, article, or system that includes the element.

Sequence numbers of the embodiments disclosed herein are meant for the sole purpose of illustrative and do not represent the advantages and disadvantages of these embodiments.

Through the above description of the foregoing embodiments, those skilled in the art can clearly understand that the above methods of the embodiments can be implemented by means of software plus a necessary general hardware platform; they certainly can also be implemented by means of hardware, but in many cases, the former is a better implementation. Based on this understanding, the essential part of the technical solution according to the present disclosure or the part that contributes to the prior art can be embodied in the form of a software product. Computer software products can be stored in a storage medium as described above (e.g., ROM/RAM, a magnetic disk, an optical disc) which includes instructions to cause a terminal device (e.g., a mobile phone, a computer, a server, an air conditioner, or a network device, etc.) to perform the methods described in the various embodiments of the present disclosure.

The foregoing description portrays merely some illustrative embodiments of the present disclosure, and are not intended to limit the patentable scope of the present disclosure. Any equivalent structural or flow transformations based on the specification and the drawing of the present disclosure, or any direct or indirect applications of the present disclosure in other related technical fields, shall all fall within the protection scope of the present disclosure.

1. A method for detecting disease probability, comprising:
collecting each datum associated with a user, and performing feature processing to each collected datum;
constructing a multi-dimensional data set according to each datum after being feature processed;
performing random sampling on the multi-dimensional data set to divide into a test set and a training set;
building a model based on the training set to obtain a regression decision tree;
testing the regression decision tree according to the test set, to calculate the disease probability of the user.

2. The method of claim 1, wherein the step of performing feature processing to each collected datum comprises:
performing feature analysis on each collected datum to determine a feature type of each datum;
when the datum is a missing value datum, performing mean imputation or multiple imputation to the missing value datum;
when the datum is an outlier datum, screening the outlier datum, and screening out the datum whose outlier is less than a preset threshold, and treating the screened datum as the missing value datum.

3. The method of claim 2, wherein the mean imputation comprises: performing interpolation using an average value, or performing interpolation using mode.

4. The method of claim 1, wherein the step of constructing a multi-dimensional data set according to each datum after being feature processed comprises:
determining feature saturation corresponding to each datum after being feature processed;
screening each datum according to the feature saturation to screen out the datum whose feature saturation reaches a preset saturation;
constructing the multi-dimensional data set based on each screened datum.

5. The method of claim 1, wherein the step of testing the regression decision tree according to the test set, to calculate the disease probability of the user comprises:
inputting the data in the test set into the regression decision tree to obtain the numerical value corresponding to the number of trees in the regression decision tree;
calculating weighted average of each numerical value with a weight value of each tree in the regression decision tree to obtain a total value of the regression decision tree;
using the total value as the disease probability of the user.

6. (canceled)

7. (canceled)

8. (canceled)

9. (canceled)

10. (canceled)

11. An apparatus for detecting disease probability, comprising:
a processor,
a memory which stores a disease probability detecting program;
wherein the processor is configured for executing the disease probability detecting program to perform following operations:
collecting each datum associated with a user, and performing feature processing to each collected datum;
constructing a multi-dimensional data set according to each datum after being feature processed;
performing random sampling on the multi-dimensional data set to divide into a test set and a training set;
building a model based on the training set to obtain a regression decision tree;
testing the regression decision tree according to the test set, to calculate the disease probability of the user.

12. The system of claim 11, wherein the processor is further configured for executing the disease probability detecting program to perform feature processing to each collected datum:
performing feature analysis on each collected datum to determine the feature type of each datum;
when the datum is a missing value datum, performing mean imputation or multiple imputation to the missing value datum;
when the datum is an outlier datum, screening the outlier datum, and screening out the datum whose outlier is less than a preset threshold, and treating the screened datum as the missing value datum.

13. The device of claim 12, wherein the mean imputation comprises: performing interpolation using an average value, or performing interpolation using mode.

14. The system of claim 11, wherein the processor is further configured for executing the disease probability detecting program to perform constructing a multi-dimensional data set according to each datum after being feature processed:
determining feature saturation corresponding to each datum being feature processed;
screening each datum according to the feature saturation to screen out the datum whose feature saturation reaches a preset saturation;
constructing the multi-dimensional data set based on each screened datum.

15. The system of claim 11, wherein the processor is further configured for executing the disease probability detecting program to perform testing the regression decision tree according to the test set, to calculate the disease probability of the user:
inputting the data in the test set into the regression decision tree to obtain the numerical value corresponding to the number of trees in the regression decision tree;
calculating weighted average of each numerical value with a weight value of each tree in the regression decision tree to obtain a total value of the regression decision tree;
using the total value as the disease probability of the user.

16. A computer-readable storage medium storing a disease probability detecting program, which when executed by a processor performs following operations:
collecting each datum associated with a user, and performing feature processing to each collected datum;
constructing a multi-dimensional data set according to each datum after being feature processed;
performing random sampling on the multi-dimensional data set to divide into a test set and a training set;
building a model based on the training set to obtain a regression decision tree;
testing the regression decision tree according to the test set, to calculate the disease probability of the user.

17. The computer-readable storage medium of claim 16, wherein when the disease probability detecting program executed by the processor, operations of performing feature processing to each collected datum are performed:
performing feature analysis on each collected datum to determine the feature type of each datum;
when the datum is a missing value datum, performing mean imputation or multiple imputation to the missing value datum;
when the datum is an outlier datum, screening the outlier datum, and screening out the datum whose outlier is less than a preset threshold, and treating the screened datum as the missing value datum.

18. The computer-readable storage medium of claim 17, wherein the mean imputation comprises: performing interpolation using an average value, or performing interpolation using mode.

19. The computer-readable storage medium of claim 16, wherein when the disease probability detecting program executed by the processor, operations of constructing a multi-dimensional data set according to each datum after being feature processed are performed:
determining feature saturation corresponding to each datum after being feature processed;
screening each datum according to the feature saturation to screen out the datum whose feature saturation reaches a preset saturation;
constructing the multi-dimensional data set based on each screened datum.

20. The computer-readable storage medium of claim 16, wherein when the disease probability detecting program executed by the processor, operations of testing the regression decision tree according to the test set, to calculate the disease probability of the user are performed:
inputting the data in the test set into the regression decision tree to obtain the numerical value corresponding to the number of trees in the regression decision tree;
calculating weighted average of each numerical value with a weight value of each tree in the regression decision tree to obtain a total value of the regression decision tree;
using the total value as the disease probability of the user.
